(19) United States
(12) Patent Application Publication
(10) Pub. No.: US 2008/0227165 A1
(43) Pub. Date: Sep. 18, 2008

(54) Process for producing conjugated fatty acid and food/drink produced by the process

(75) Inventors: Naomi Mizusawa, Tokyo (JP); Masashi Sakai, Tokyo (JP); Satoshi Kudo, Tokyo (JP); Yukio Shirasawa, Tokyo (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: KABUSHIKI KAISHA YAKULT HONSHA, Tokyo (JP)

(21) Appl. No.: 12/082,917

(22) Filed: Apr. 15, 2008

Related U.S. Application Data
(63) Continuation of application No. 10/511,230, filed on May 23, 2005.

(30) Foreign Application Priority Data
Apr. 12, 2002 (JP) 2002-110773
Oct. 16, 2002 (JP) 2002-301286

(51) Int. Cl. C12P 7/64; C07C 57/12

(52) U.S. Cl. 435/134; 554/230

(57) ABSTRACT

A conjugated fatty acid such as the cis-9, trans-11 isomer of conjugated linoleic acid having high bioactivities is selectively and efficiently produced by conjugating an unsaturated fatty acid having at least two double bonds with the use of viral cells, dead cells or a cell extract of at least one bacterium selected from the group consisting of Lactobacillus oris, Lactobacillus pontis, Lactobacillus panis, Bifidobacterium breve, Bifidobacterium bifidum, Bifidobacterium infantis and Bifidobacterium pseudocatenulatum, or an enzyme derived from the bacterium.

### CROSS-REFERENCE TO RELATED APPLICATIONS

This application is a continuation application of application Ser. No. 10/511,230 filed May 23, 2005, which is a U.S. national phase application under 35 USC 371 of International Application PCT/JP03/04633 filed Apr. 11, 2003.

### TECHNICAL FIELD

The present invention relates to a process for producing a specific conjugated fatty acid, specifically for the process using at least one bacterium selected from Lactobacillus oris, Lactobacillus pontis, Lactobacillus panis, Bifidobacterium breve, Bifidobacterium bifidum, Bifidobacterium infantis and Bifidobacterium pseudocatenulatum. The invention also rerates to a food/drink containing a conjugated fatty acid produced by the said process.

### BACKGROUND ART

Conjugated fatty acids are fatty acids each having double bonds at adjacent carbons with the interposition of a single bond. Among them, conjugated linoleic acids each containing 18 carbon atoms and having one conjugated diene in their molecule have been found to have various bioactivities.

The conjugated linoleic acids are industrially produced, for example, by alkali conjugation, in which an oil or fat containing linoleic acid, or free linoleic acid is conjugated in an organic solvent such as ethylene glycol.

In the alkali conjugation, an unsaturated fatty acid and excess alkali are generally heated to 150° C. or higher in an organic solvent. The resulting conjugated fatty acid is a mixture of isomers having different bonding sites and/or configurations of double bonds. Linoleic acid as the raw material, for example, yields the cis-9, trans-11 isomer, trans-9, cis-11 isomer and trans-10, cis-12 isomer of conjugated linoleic acid, as well as some other positional isomers and/or geometric isomers.

The bioactivities of the conjugated fatty acid are activities as a mixture of conjugated fatty acids. A technique for producing a specific conjugated fatty acid, if developed, has high academic and industrial importance. The alkali conjugation invites cyclization and other side reactions to decrease the yield of the conjugated fatty acid and to inhibit its purification.

Certain microorganisms and enzymes thereof have been reported to produce conjugated fatty acids. It has been reported, for example, that rumen bacteria produce conjugated fatty acids from a polyvalent unsaturated fatty acid (Shorland F. B., et al., Nature, vol. 175, p. 1129, 1955), Treponema (Yokoyama, et al., J. Bacteriology, vol. 107, pp. 519-527, 1971), Butyrivibrio fibrisolvens (Kepler C. R., et al., J. Biol. Chem., vol. 242, pp. 5686-92, 1967), Propionibacterium freudenreichii (Jiang J., Doctoral thesis, Swedish Uni. Uppsala, 1998), and ten and several percent of various pulmonary-pathogenic microorganisms (Jack C. I. A., et al., Clinica Chlimica Acts., vol 224, pp. 139-4, 1994) have a capability of isomerizing linoleic acid, and that these microorganisms produce conjugated linoleic acids mainly comprising the cis-9, trans-11 isomer.

Ruminant animals the cis-9, trans-11 isomer of conjugated linoleic acid in vivo, and dairy products and livestock meat contain a large amount of the cis-9, trans-11 isomer of conjugated linoleic acid. Thus, the cis-9, trans-11 isomer of conjugated linoleic acid is believed to be a linoleic acid which human beings frequently take in general diet. A variety of investigations have been made on bioactivities of this isomer to find that the isomer has a defensive action against various cancers (Ha, Y. L., et al., Cancer Research vol. 50, p 1097-1101, 1990, Ip C., et al., Cancer Research vol. 51, pp. 6118-6124).

As is described above, a product enriched for the cis-9, trans-11 isomer of conjugated linoleic acid having a promising anticarcinogenic activity is expected to be produced by using a microorganism. No microorganism that produces a sufficient amount of a conjugated linoleic acid with less by-products, however, has yet been found.

Lactic acid bacteria and bacteria belonging to the genus Bifidobacterium are known as useful microorganisms for food. The bacteria belonging to the genus Bifidobacterium colonize in human colon, have various useful actions such as improvement in feculent and inhibition of enteral putrefaction and have been used in, for example, fermented milk.

The bacteria belonging to the genus Bifidobacterium are obligatory anaerobic and tend to die in the presence of oxygen or at a low pH. Thus, investigations using the bacteria belonging to the genus Bifidobacterium require an appropriate working environment and skilled operations. Thus, there has been little investigation on production of substances, including production of a conjugated linoleic acid, using the bacteria belonging to the genus Bifidobacterium.

Korean Patent Publication KR-A-2001-0089858 (published on Oct. 12, 2001) proposes a fermented milk composition containing 0.001-5 wt % of a conjugated linoleic acid using a lactic acid bacterium, and a production process thereof.

As is described above, the alkali conjugation technique requires the reaction at elevated temperatures, often invites side reactions, is difficult to yield a specific conjugated fatty acid and requires a complicated purification step thereafter. The conversion reaction using a microorganism by-produces an all trans isomer of conjugated linoleic acid and produces conjugated linoleic acids in an insufficient total production amount.

The above-mentioned Korean Patent Publication discloses Lactobacillus acidophilus, Lactobacillus casei, Bifidobacterium longum, Bifidobacterium adolescentis, Bifidobacterium breve and Bifidobacterium infantis as the lactic acid bacteria producing a conjugated fatty acid in fermented milk compositions obtained by the use of such lactic acid bacteria. The publication also indicates that the production capability varies from strain to strain even in one species, and this is also indicated by investigations to achieve the present invention. No bacterial strain which produces 10 or more of the product based on 100 of the substrate linoleic acid has been obtained, although some bacterial strains produces 5 or more.

To solve these problems, an object of the present invention is to conjugate an unsaturated fatty acid having at least two double bonds, and specifically to provide a process for efficiently and selectively preparing an enriched highly bioactive cis-9, trans-11 isomer of conjugated linoleic acid alone from linoleic acid. Another object of the present invention is to provide a bacterium capable of efficiently producing an enriched highly bioactive cis-9, trans-11 isomer of conjugated linoleic acid alone from linoleic acid, and a food/drink containing a conjugated fatty acid.

### DISCLOSURE OF INVENTION

After intensive investigations to find a process for producing a conjugated linoleic acid with the use of a bacterium, the present inventors have found that certain bacteria belonging to the genus Lactobacillus and/or bacteria belonging to the genus Bifidobacterium are capable of conjugating, namely, are able to transfer the position of double bonds in the molecule of an unsaturated fatty acid and to isomerize into an isomer in which the double bonds are conjugated with each other.

According to the present invention, the above-mentioned objects can be achieved by a process for producing a conjugated fatty acid comprising a step of conjugating an unsaturated fatty acid having at least two double bonds with the use of viral cells, dead cells or a cell extract of a bacterium having conjugation capability and belonging to the genus Lactobacillus and/or belonging to the genus Bifidobacterium, or an enzyme derived from the bacterium.

More specifically, the unsaturated fatty acid having at least two double bonds is conjugated by using viral cells, dead cells or a cell extract of at least one bacterium having conjugation capability selected from the group consisting of Lactobacillus oris, Lactobacillus pontis, Lactobacillus panis, Bifidobacterium breve, Bifidobacterium bifidum, Bifidobacterium infantis and Bifidobacterium pseudocatenulatum, or an enzyme derived from the bacterium.

The process according to the present invention conjugates an unsaturated fatty acid having at least two double bonds with the use of viral cells, dead cells or a cell extract of a bacterium belonging to the genus Lactobacillus and/or a bacterium belonging to the genus Bifidobacterium, or an enzyme derived from the bacterium. Thus, the process can produce a conjugated fatty acid typified by the cis-9, trans-11 isomer of conjugated linoleic acid which is believed to have high bioactivities selectively in a high content and high yield.

Lactobacillus oris for use in the process for producing a conjugated fatty acid according to the present invention is a bacterium isolated from human saliva (Farrow J. A. E. et al., Int. J. Syst. Bacteriol. vol. 38, p. 116, 1988). Examples of the strain thereof are Lactobacillus oris NCDO 2160 (ATCC 49062), NCDO 2162, NCDO 2163 and NCDO 2164, of which NCDO 2160 (ATCC 49062) is preferred.

Lactobacillus pontis is a bacterium isolated form rye bread leaven (Vogel, R. F., et al., Int. J. Syst. Bacteriol., vol. 44, pp. 223-229, 1994). Examples of the strain thereof are Lactobacillus pontis ATCC 51518 and ATCC 51519, of which ATCC 51518 is preferably used.

Lactobacillus panis is a bacterium isolated from rye bread leaven (Sirohmar, W., Diekmann, H., Z. Lebensm. Unters. Forsch, vol. 194, pp. 536-540, 1992). Examples of the strain thereof include Lactobacillus panis JCM 11053, of which JCM 11053 is preferably used.

Bifidobacterium breve for use in the process for producing a conjugated fatty acid according to the present invention is a strain isolated from faeces and vaginalis of infants and suckling calves. Examples of the strain thereof are Bifidobacterium breve ATCC 15698, ATCC 15701 and YIT 10001, of which Bifidobacterium breve YIT 10001 is an excellent strain that can efficiently produces the cis-9, trans-11 isomer of conjugated linoleic acid in a high content. Bifidobacterium breve YIT 10001 was deposited as FERM P-18459 at International Patent Organism Depositary National Institute of Advanced Industrial Science and Technology on Aug. 14, 2001 and was transferred to International Deposit FERM BP-8205 on Oct. 11, 2002. Bifidobacterium breve YIT 1001 strain has a very high productivity of a conjugated linoleic acid and is preferred. This strain is capable of producing a conjugated linoleic acid in an amount of at least 0.5 mg/5-mL in a medium by preculturing in GAM broth (a product of Nissui Pharmaceutical Co., Ltd.) containing a complex between linoleic acid and BSA for at least 24 hours, inoculating 3.8 wt % of the cultured bacterium belonging to the genus Bifidobacterium to a milk medium containing a complex between linoleic acid and BSA and cultivating the bacterium with shaking for 144 hours. Such a strain having the excellent productivity can produce a conjugated linoleic acid to such an extent to expect bioactivities in a fermented food such as fermented milk when a food material such as milk material containing linoleic acid is fermented using the strain. Thus, the strain is typically suitable in, for example, food production with good operability.

Bifidobacterium infantis is a strain isolated from the faeces of infants. An example of the strain thereof is Bifidobacterium infantis ATCC 15702.

Bifidobacterium bifidum is a strain isolated from the faeces and vaginalis of adults, infants and suckling calves. An example of the strain thereof is Bifidobacterium bifidum YIT 4007 (FERM BP-791).

Bifidobacterium pseudocatenulatum is a strain isolated from sewerage, the faeces of infants and faeces of suckling calves. An example of the strain thereof is Bifidobacterium pseudocatenulatum ATCC 27919.

The unsaturated fatty acid having at least two double bonds for use as a raw material in the present invention is not specifically limited and can be any of, for example, linoleic acid, linolenic acid, arachidonic acid, eicosapentaenoic acid and docosahexaenoic acid. Among them, linoleic acid is preferably used as the raw material to yield a conjugated linoleic acid. In this case, a specific isomer which has been reported to have bioactivities is specifically and efficiently produced with very trace amounts of other isomers (by-products).

The unsaturated fatty acid may be in the form of, for example, a salt or ester. Examples of the salt are salts of alkali metals, such as sodium salt and potassium salt; salts of alkaline earth metals, such as calcium salt and magnesium salt; and ammonium salts. Examples of the ester are methyl ester and ethyl ester. In addition, other lipids (phospholipids and glycolipids), monoglycerides, diglycerides and triglycerides each containing the unsaturated fatty acid can also be used.

Naturally occurring oils and fats can also be used as the raw material. Examples of those containing a large amount of linoleic acid in the molecule are naturally occurring oils and fats derived from vegetables, such as safflower oil, cotton seed oil, soybean oil, sunflower seed oil, corn oil, peanut oil, rice bran oil, linseed oil and cacao butter. Examples of those containing a large amount of linolenic acid in the molecule are naturally occurring oils and fats derived from animals, such as sardine oil, herring oil and cod oil. Products of these decomposed by the action of a lipase can also be used as the raw material.

The bacterium belonging to the genus Lactobacillus and/or bacterium belonging to the genus Bifidobacterium is preferably inoculated and cultured in a culture medium containing an unsaturated fatty acid having at least two double bonds in the present invention. A milk medium is preferably used as the culture medium.

The unsaturated fatty acid can be conjugated with the use of a bacterium belonging to the genus Lactobacillus and/or a bacterium belonging to the genus Bifidobacterium, preferably at least one selected from the group consisting of Lactobacillus oris, Lactobacillus pontis, Lactobacillus panis, Bifidobacterium breve, Bifidobacterium bifidum, Bifidobacterium infantis and Bifidobacterium pseudocatenulatum by any technique to produce a conjugated fatty acid in the present invention. Examples of such techniques are a technique of cultivating the bacterium in a growth medium containing the material unsaturated fatty acid to thereby produce a conjugated fatty acid directly; and a technique of cultivating the bacterium by any means, collecting and washing the resulting cells and adding the washed cells to a solution containing the material unsaturated fatty acid and allowing the cells to react to thereby produce a conjugated fatty acid. Not only viral cells of the bacterium, but also dead cells or a cell extract of the bacterium or an enzyme extracted from the cells can also be used for the reaction in a solution containing the unsaturated fatty acid.

Any of media generally used for the growth of bacteria belonging to the genus Lactobacillus and/or bacteria belonging to the genus Bifidobacterium, or a milk medium containing milk can be used as the culture medium for cultivating the bacterium belonging to the genus Lactobacillus and/or bacterium belonging to the genus Bifidobacterium, such as Lactobacillus oris, Lactobacillus pontis, Lactobacillus panis, Bifidobacterium breve, Bifidobacterium bifidum, Bifidobacterium infantis and Bifidobacterium pseudocatenulatum. The unsaturated fatty acid is preferably treated in a culture medium containing milk.

When a generally used growth medium such as MRS medium or GAM broth is used, the raw material oil such as linoleic acid is not dispersed in the medium and the treatment is carried out inefficiently. Thus, the addition of a lipid-binding protein such as BSA (bovine serum albumin) or a surfactant, and/or homogenization under severe conditions is required. By using a milk medium, however, the material oil can be relatively easily homogenized without the addition of BSA, thus improving the operability and treatment efficiency and avoiding an increased cost due to the addition typically of BSA. When the present invention is applied to the production of a food containing a conjugated fatty acid, it is preferred to use a milk medium that can carry out the reaction efficiently even if the medium is free from BSA to thereby produce a fermented milk. This is because the lipid-binding protein such as BSA and the surfactant affects the flavor.

Such a milk medium can disperse the material oil excellently, probably because of the effects of protein components contained in the milk. The “milk” as used in the present description refers to lactoprotein-containing substances including raw milk, skim milk powder, whole milk powder or fresh cream of an animal milk such as cow's milk or goat's milk; as well as a vegetable milk such as soybean milk, almond milk or coconut milk.

To produce a conjugated linoleic acid, the bacterium belonging to the genus Lactobacillus and/or the bacterium belonging to the genus Bifidobacterium is preferably precultured in a culture medium containing a complex between linoleic acid and at least one material selected from BSA, lipid-binding proteins and surfactants, although such a technique has the above-mentioned problems typically of cost. This configuration effectively increases the production of the conjugated linoleic acid in the main culture. The preculture is carried out for preferably 12 hours or more and more preferably 20 hours or more.

Washed cells, a cell powder, dead cells or a cell extract of the bacterium belonging to the genus Lactobacillus and/or the bacterium belonging to the genus Bifidobacterium such as Lactobacillus oris, Lactobacillus pontis, Lactobacillus panis, Bifidobacterium breve, Bifidobacterium bifidum, Bifidobacterium infantis and Bifidobacterium pseudocatenulatum for use in the reaction can be prepared according to a conventional procedure. The washed cells, for example, can be prepared by washing cultured cells with physiological saline or buffer. The cell powder can be prepared according to a drying technique such as freeze-drying or spray drying.

Examples of the techniques for preparing dead cells are a technique of allowing a cell-wall digesting enzyme, a technique of treating at a low osmosis, a technique of freeze-thawing, a technique of treating under high pressure, a technique of pulverization and a technique of heating. Among them, the technique of heating cells to allow autolysis can treat a large amount of cells at low cost and is preferred. The cell extract can be prepared by adding an appropriate solvent typically to the above-prepared washed cells, cell powder or dead cells and subjecting the mixture to centrifugal separation to yield the cell extract as a supernatant.

Examples of the conjugation process to yield a conjugated fatty acid with the use of at least one bacterium having conjugation capability selected from the group consisting of Lactobacillus oris, Lactobacillus pontis, Lactobacillus panis, Bifidobacterium breve, Bifidobacterium bifidum, Bifidobacterium infantis and Bifidobacterium pseudocatenulatum are the following processes (a) to (e):

(a) a process of cultivating the bacterium in a growth medium or milk medium (fermented milk) to conjugate an unsaturated fatty acid in the form of fermentative production;

(b) a process of conjugating an unsaturated fatty acid with the use of washed cells of the bacterium;

(c) a process of conjugating an unsaturated fatty acid with the use of a cell powder of the bacterium;

(d) a process of conjugating an unsaturated fatty acid with the use of dead cells of the bacterium; and

(e) a process of conjugating an unsaturated fatty acid with the use of a cell extract of the bacterium.

The processes (a) to (e) will be illustrated in detail below.

(a) Process of Cultivating the Bacterium in a Growth Medium or Fermented Milk to Conjugate an Unsaturated Fatty Acid in the Form of Fermentative Production:

The basic operation of conjugation in a growth medium or fermented milk may be carried out according to a conventional procedure. The following starter is preferably used.

Initially, an unsaturated fatty acid such as linoleic acid is added to a lactic acid bacterium growth medium such as MRS medium (LACTOBACILLI MRS BROTH, a product of DIFCO) or a growth medium for anaerobe such as GAM medium (GAM broth, a product of Nissui Pharmaceutical Co., Ltd.) to induce the conversion ability efficiently in cell preparation. The concentration herein is in the range of 0% to 0.2% and preferably 0.05% to 0.1%.

This cultivation procedure is preferably stopped at pH 3.5 to 5.5, and typically preferably at pH 4.5 to 5.5. If the cultivation is continued to pH 3.5 or below, the capability of conjugating may decrease due to overgrowth of the bacterium. If the cultivation is continued at pH 5.5 or higher, no sufficient amount of cells to conjugate is obtained.

The resulting cultured mixture is preferably used as a starter. The starter is inoculated in an amount of preferably 0.5% to 8%, and typically preferably about 1% to 4% of a culture medium for the preparation of fermented milk. The amount of the raw material in the culture medium is preferably in the range of about 0.02% to 0.8% and typically preferably 0.1% to 0.2%. A culture temperature is in the range of about 20° C. to 40° C., and preferably 28° C. to 37° C.

For more efficiently yielding a conjugated fatty acid, the cultivation is preferably stopped at pH 3.5 to 5.5 and more preferably at pH 4.5 to 5.5. The target conjugated fatty acid can also be prepared in a neutralization culture.
(b) Process of Conjugating an Unsaturated Fatty Acid with the Use of Washed Cells of the Bacterium:

Cells cultivated under the same conditions in the preparation of the starter in Process (a) are washed with physiological saline, collected and suspended in a buffer. Aqueous solutions that can maintain an appropriate pH are used as the buffer in which the cells are suspended and for the reaction of the washed cells. A 0.1 to 1.0 M phosphate buffer, for example, is preferably used. The pH of the buffer is 5.0 to 7.5 and preferably 6.0 to 7.0. The concentration of cells in the cell suspension is in the range of 0.025% to 0.25% and preferably 0.025% to 0.1% in terms of wet weight. The raw material has been mixed with bovine serum albumin (BSA) and is added to the reaction mixture in an amount of preferably in the range of about 0.1% to 4.0% and typically preferably 0.3% to 1.0%.

The amount of BSA is preferably about one fifth of that of the raw material. The temperature in the conversion reaction by the washed cells is 20° C. to 52° C. and preferably 32° C. to 37° C. An appropriate time for conversion and production is 1 to 96 hours and preferably 24 to 72 hours. Cells cultivated while neutralizing the pH can also be used.

(c) Process of Conjugating an Unsaturated Fatty Acid with the Use of a Cell Powder of the Bacterium:

The cell powder of the bacterium can be prepared by cultivating the bacterium under the same conditions as in the preparation of the starter in Process (a), drying and powdering the resulting cells of the bacterium. The cells can be dried, for example, by freeze-drying or spray drying. The conversion reaction after the preparation of the cell powder may be carried out under the same conditions as in the reaction of washed cells in Process (b).

(d) Process of Conjugating an Unsaturated Fatty Acid with the Use of Dead Cells of the Bacterium:

The cells of the bacterium can be prepared by cultivating the bacterium under the same conditions as in the preparation of the starter in Process (a) and breaking the cell walls of the cells. The cell wall can be broken, for example, by any of a process of treating with an cell wall digestive enzyme, a process of suspending the cells in a solvent and treating the suspension at a low osmosis, a process of freezing and thawing, a process of treating under high pressure, a process of pulverization, and a process of heating. The conversion reaction after the preparation of the cell wall digestive product may be carried out under the same conditions as in the reaction for the washed cells in Process (b).

(e) Process of Conjugating an Unsaturated Fatty Acid with the Use of a Cell Extract of the Bacterium:

The cell extract of the bacterium can be prepared, for example, by extracting washed cells, cell powder or dead cells of the bacterium prepared by the procedure of Process (b), (c) or (d) with an appropriate solvent and removing the residue typically by centrifugal separation. The conversion reaction after the preparation of the cell extract may be carried out under the same conditions as in the reaction for the washed cells in Process (b).

The amounts of individual fatty acids in the fatty acid composition of the reaction mixture or cultured mixture obtained by the action of the bacterium, for example, by means of above-mentioned Process (a), (b), (c) or (d) can be determined by calculation based on the ratios of the areas of the individual fatty acids to that of an internal standard measured by gas chromatographic analysis. When the conjugated linoleic acids are the target compounds, gas chromatography may be carried out under the conditions shown in Table 1.

TABLE 1
GC Analytical Condition

| GC system | GC-358 (a product of GL Sciences Inc.) |
|---|---|
| | GC14A (a product of Shimadzu Corporation) |
| Colum | DB-23 (ID 0.25 mm × 30 m) |
| Oven | 160° C. → 220° C. (at 2° C./min) |
| Inj. Temp. | 250° C. |
| Det. Temp. | 250° C. |
| Carrier Gas | N₂ (50 mL/min) |
| Detector | FID |
| Sample Size | 1 μL in hexane |
| Sprit | 1/100 |

The conjugated fatty acid prepared according to the present invention can be administered in the form of, for example, pharmaceutical preparations, foods or cosmetics. In the case of a pharmaceutical preparation or a nutritional supplementary food which aims at bioactivities of a conjugated linoleic acid, the conjugated linoleic acid can be orally administered in the dosage form of a solid preparation such as a capsule, granule, tablet or powder, or a liquid preparation such as syrup. The conjugated fatty acid can also be administered non-orally in the dosage form typically of an injection, skin external preparation or rectal preparation, instead of such an oral preparation.

Each preparation can be produced with the use of additives. Examples of such additives are excipients or fillers such as lactose, starch, crystalline cellulose, calcium lactate, magnesium aluminometasilicate and silicic anhydride; binders such as sucrose, hydroxypropyl cellulose and polyvinylpyrrolidone; disintegrators such as carboxymethylcellulose and calcium carboxymethylcellulose; lubricants such as magnesium stearate, talc, monoglyceride and sucrose fatty acid esters; as well as other ingredients that are acceptable typically as pharmaceutical preparations and/or food.

When used in the form of a general food (in the form of an “apparent food”) expecting similar bioactivities, such a food may be produced according to a conventional procedure by adding the conjugated fatty acid obtained by the process of the present invention to a drink/food such as an oil, tablet-type sweets, fermented milk, candies, flavoring materials or dried food sprinkled over rice as intact or after appropriate purification. When used as a fermented food, the fermented food can be produced by adding a fatty acid having at least two double bonds to a raw material for fermentation and fermenting (cultivating) the raw material by the action of a fermentative microorganism. It is preferred that milk containing linoleic acid is fermented to yield a fermented milk, since the resulting fermented milk can easily contain a large amount of the cis-9, trans-11 isomer of conjugated linoleic acid specifically, as described above.

A composition containing conjugated linoleic acid mainly comprising the cis-9, trans-11 isomer of conjugated linoleic acid can be obtained according to the present invention by using at least one selected from the group consisting of Lactobacillus oris ATCC 49062, Lactobacillus pontis ATCC 51518, Lactobacillus pontis ATCC 51519, Lactobacillus panis JCM 11053, Bifidobacterium breve YIT 10001, Bifidobacterium breve ATCC 15698, Bifidobacterium breve ATCC 15701, Bifidobacterium bifidum YIT 4007, Bifidobacterium infantis ATCC 15702 and Bifidobacterium pseudocatenulatum ATCC 27919. The “composition of conjugated linoleic acid enriched for the cis-9, trans-11 isomer” used in the present invention refers to a composition of conjugated linoleic acid in which other conjugated linoleic acid isomers than the cis-9, trans-11 isomer of conjugated linoleic acid occupies 10% or less of the total isomers of conjugated linoleic acid. Such a composition has promising bioactivities such as anticancer activity even in a small amount and is excellent in taste, flavor and operability when added typically to a food. The ratio of the other isomers than the cis-9, trans-11 isomer of conjugated linoleic acid is more preferably 4% or less in the composition.

The present invention further provides a food/drink containing a conjugated fatty acid obtained by the process according to the present invention.

The “fermented milk” refers to and includes lactic drinks containing viral cells, such as fermented milk, milk products, lactic acid bacteria beverages; and lactic drinks containing sterilized fermented milk, as specified in the Ministerial Ordinance concerning Compositional Standards, etc. for Milk and Milk Products, the Ministry of Health, Labor and Welfare of Japan, as well as kefir. In the fermentation, other microorganisms including bacteria belonging to the genus Lactobacillus, such as Lactobacillus casei, Lactobacillus acidophilus, Lactobacillus gasseri Lactobacillus zeae, Lactobacillus johnsonii, Lactobacillus delbrueckii (ss. bulgaricus) and Lactobacillus delbrueckii (ss. delbrueckii); bacteria belonging to the genus Streptococcus, such as Streptococcus thermophilus; bacteria belonging to the genus Lactococcus, such as Lactococcus lactis (ss. lactis), Lactococcus lactis (ss. cremoris), Lactococcus plantarum and Lactococcus raffinolactis; bacteria belonging to the genus Leuconostoc, such as Leuconostoc mesenteroides and Leuconostoc lactis; and bacteria belonging to the genus Enterococcus, such as Enterococcus feacalis and Enterococcus faecium.

Other microorganisms including bacteria belonging to the genus Bifidobacterium, such as Bifidobacterium breve, Bifidobacterium bifidum, Bifidobacterium longum and Bifidobacterium animalis, as well as yeasts can also be used. Each of these can be used alone or in combination.

These foods may further comprise other food materials such as carbohydrates, emulsifiers, thickeners, sweeteners, acidulants and fruit juice. Examples thereof are saccharides such as sucrose, isomerized sugar, glucose, fructose, paratinose, trehalose, lactose and xylose; sugar alcohols such as sorbitol, xylitol, erythritol, lactitol, paratinit, reduced starch syrup and reduced maltose syrup; emulsifiers such as sucrose fatty acid esters, glycerol fatty acid esters and lecithin; and thickeners (thickening stabilizers) such as carrageenan, Cyamoposis Gum, xanthan gum and Locust bean gum. In addition, vitamins such as vitamin A, and B vitamins; and minerals such as calcium, iron, manganese and zinc may also be incorporated.

The conjugated fatty acid produced by the process of the present invention can be appropriately incorporated into such pharmaceutical preparations and foods. When the bioactivities of the conjugated fatty acid are required, the conjugated fatty acid may be incorporated in such an amount as to exhibit the bioactivities and avoid problems such as overdose, namely, such an amount as to take the conjugated fatty acid in an amount in the range of about 10 mg to 1,000 mg per day.

### BRIEF DESCRIPTION OF THE DRAWINGS

FIG. 1 shows gas chromatographic charts of conjugated linoleic acid isomers produced by Lactobacillus oris, in which graphs a, b and c are charts of a linoleic acid standard, a conjugated linoleic acid standard and a reaction mixture by the action of Lactobacillus oris, respectively. In the charts, Peak A shows the internal standard (17:0), Peak B linoleic acid, Peak C the cis-9, trans-11 isomer of conjugated linoleic acid, Peak D the trans-10, cis-12 isomer of conjugated linoleic acid, Peak E all-cis isomer of conjugated linoleic acid, and Peak F all-trans isomer of conjugated linoleic acid. The crowd of Peaks C, D, E and F shows the total isomers of conjugated linoleic acid.

### BEST MODE FOR CARRYING OUT THE INVENTION

The present invention will now be explained with reference to some examples below, which are not intended to limit the scope of the present invention.

### Example 1

### Screening of Bacteria Belonging to the Genus Lactobacillus and Producing a Conjugated Fatty Acid

In 1 mL of a 100 mM phosphate buffer (pH 6.5) were dissolved 50 mg of linoleic acid and 10 mg of BSA to yield a solution of a complex between linoleic acid and BSA.

Bacteria belonging to the genus Lactobacillus were inoculated to 15 mL of MRS medium (LACTOBACILLI MRS BROTH, a product of DIFCO) containing 0.07% of linoleic acid and cultivated at 28° C. for 20 hours with shaking at 120 rpm. The cultured mixture had a pH of 4.7.

The cultured mixture was subjected to centrifugal separation to collect cells, and the cells were washed twice with physiological saline to yield washed cells. The washed cells were mixed with 100 μL of the solution of a complex between linoleic acid and BSA, and 0.9 mL of a 100 mM phosphate buffer (pH 6.5), and the mixture was reacted in an oxygen-impermeable plastic bag whose atmosphere was maintained anaerobic by using an agent for oxygen absorbing and carbon dioxide gas generating agent (Anaero Pack, a product of Mitsubishi Gas Chemical Company, Inc.) at 37° C., 120 rpm for 24 hours.

The resulting reaction mixture was mixed with 1 mg of an internal standard (HEPTA-DECANOIC ACID), extracted by a Bligh-Dyer method, converted into a methyl ester (standing still in a 4% solution of hydrochloric acid in methanol at room temperature for 30 minutes) and analyzed on fatty acids by gas chromatography.

Peaks of the conjugated linoleic acids were determined on the basis of the retention time of the standard (CLA 80, a product of RINORU OIL MILLS CO., LTD.), and their relative values were determined by calculation, taking linoleic acid added as the substrate as 100. The results in Table 2 show that Lactobacillus oris produces a conjugated linoleic acid. This strain, Lactobacillus oris NCDO 2160, has been registered as ATCC 49062 to the American Type Culture Collection (ATCC).

TABLE 2

| | Conjugated linolic acid level, |
|---|---|
| Strain | taking substrate linoleic acid as 100 |
| Control without bacterium | 0.1 |
| Lactobacillus acidophilus | 0.0 |
| Lactobacillus brevis | 0.1 |
| Lactobacillus brevis | 0.3 |
| Lactobacillus casei | 0.1 |
| Lactobacillus casei | 0.1 |
| Lactobacillus gasseri | 0.3 |
| Lactobacillus johnsonii | 0.0 |
| Lactobacillus mali | 0.0 |
| Lactobacillus oris NCD 2160 | 14.2 |
| Lactobacillus reuderi | 0.0 |
| Lactobacillus reuderi | 0.0 |
| Lactobacillus rhamnosus | 0.0 |
| Lactobacillus rhamnosus | 0.0 |
| Lactobacillus sake | 0.4 |

### Example 2

### Screening of Bacteria Belonging to the Genus Bifidobacterium and Producing a Conjugated Fatty Acid

In 1 mL of a 100 mM phosphate buffer (pH 6.5) were dissolved 50 mg of linoleic acid and 10 mg of BSA to yield a solution of a complex between linoleic acid and BSA. A total of 200 μL of the solution of a complex between linoleic acid and BSA was added to 15 mL of GAM broth (a product of Nissui Pharmaceutical Co., Ltd.), and fifteen strains of bacterium belonging to the genus Bifidobacterium were inoculated into the mixture, respectively, and cultivated at 35° C. for 48 hours with shaking at 120 rpm, to yield cultured media of bacteria belonging to the genus Bifidobacterium.

To 5 mL of 10% skim milk medium (supplemented with 1% of glucose and 0.1% of soybean peptide) dispensed in 15-mL test tubes with a cap were added 100 μL (linoleic acid content: 5 mg) of the solution of a complex between linoleic acid and BSA and 200 μL of the above-prepared cultured media of the bacteria belonging to the genus Bifidobacterium, respectively, and the cap was closed. The media were cultivated at 35° C. for 144 hours with shaking at 120 rpm to yield fermented milk products. The resulting fermented milk was mixed with 1 mg of an internal standard (HEPTADECANOIC ACID), extracted according to a Bligh-Dyer method, converted into a methyl ester (standing still in a 4% solution of hydrochloric acid in methanol at room temperature for 30 minutes) and analyzed on fatty acids by gas chromatography. The results are shown in Table 3 below.

TABLE 3
Fermented Milk Cultivated for 144 hours (Induction: linoleic acid) (Unit (mg/5-mL)

| | | | | | Conjugated | | | | | |
|---|---|---|---|---|---|---|---|---|---|---|
| | | External | pH | | linoleic acid | | | | | |
| | YIT | registry | after | Linoleic | | | All | Hydroxy acid | | |
| Strain | NO. | No. | cultivation | acid | C9-t11 | T10-C12 | trans | HOA 1 | HOA 2 | Others |
| B. adolescentis | 4011 | ATCC 15703 | 3.6 | 1.08 | 0.00 | 0.00 | 0.00 | 0.08 | 1.32 | 0.56 |
| B. adolescentis | 4087 | JCM 7042 | 3.6 | 1.04 | 0.00 | 0.01 | 0.00 | 0.10 | 1.57 | 0.33 |
| B. bifidum | 4007 | FERM BP-791 | 3.5 | 3.84 | 0.02 | 0.00 | 0.00 | 0.07 | 0.18 | 0.69 |
| B. bifidum | 4013 | | 5.2 | 1.41 | 0.00 | 0.00 | 0.00 | 0.00 | 0.00 | 0.85 |
| B. breve | 4064 | | 3.6 | 3.11 | 0.00 | 0.00 | 0.00 | 0.00 | 0.00 | 0.76 |
| B. breve | 4065 | | 3.6 | 1.93 | 0.00 | 0.00 | 0.00 | 0.00 | 0.00 | 0.46 |
| B. breve | 10001 | FERM BP-8205 | 3.5 | 0.61 | 0.57 | 0.00 | 0.02 | 0.00 | 0.00 | 0.31 |
| B. catenulatum | 4016 | ATCC 27539 | 3.3 | 2.99 | 0.00 | 0.00 | 0.00 | 0.06 | 0.49 | 0.66 |
| B. catenulatum | 4118 | JCM 7130 | 4.4 | 3.99 | 0.00 | 0.00 | 0.00 | 0.00 | 0.00 | 0.73 |
| B. infantis | 4018 | ATCC 15697 | 6.0 | 4.28 | 0.00 | 0.00 | 0.00 | 0.00 | 0.00 | 0.89 |
| B. infantis | 4019 | ATCC 15702 | 4.7 | 0.87 | 0.05 | 0.00 | 0.00 | 0.00 | 0.00 | 0.52 |
| B. lactis | 4121 | DSM 10140 | 4.3 | 3.81 | 0.00 | 0.00 | 0.00 | 0.05 | 0.07 | 0.61 |
| B. longum | 4021 | ATCC 15707 | 3.8 | 2.81 | 0.00 | 0.00 | 0.00 | 0.06 | 0.56 | 0.70 |
| B. longum | 4037 | ATCC 15708 | 3.6 | 0.99 | 0.00 | 0.00 | 0.00 | 0.00 | 0.00 | 0.27 |
| B. pseudocatennulatum | 4072 | ATCC 27919 | 3.7 | 4.02 | 0.02 | 0.00 | 0.00 | 0.00 | 0.00 | 0.99 |

Peaks of conjugated linoleic acids were determined and analyzed on the basis of the pretension time of a standard (CLA 80, a product of RINORU OIL MILLS CO., LTD.). As a result, Bifidobacterium breve YIT 10001 (FERM BP-8205), Bifidobacterium infantis ATCC 15702, Bifidobacterium bifidum FERM BP-791 and Bifidobacterium pseudocatenulatum ATCC 27919 were found to produce a conjugated linoleic acid.

Among them, Bifidobacterium breve YIT 10001 (FERM BP-8205) converts 11.4% (=0.57 mg/5 mg×100) of the added linoleic acid (5 mg), and most of (96% or more (0.5710159×100) of the produced conjugated linoleic acids is the cis-9, trans-11 isomer of conjugated linoleic acid. However, Bifidobacterium breve YIT 4046 and Bifidobacterium breve YIT 4065 have no activity for producing a conjugated linoleic acid, although they belong to the species Bifidobacterium breve, showing that the production activity for a conjugated linoleic acid is present in a specific strain of Bifidobacterium breve.

### Example 3

### Identification of Isomers in Conjugated Fatty Acids Produced by Bacterium Belonging to the Genus Lactobacillus

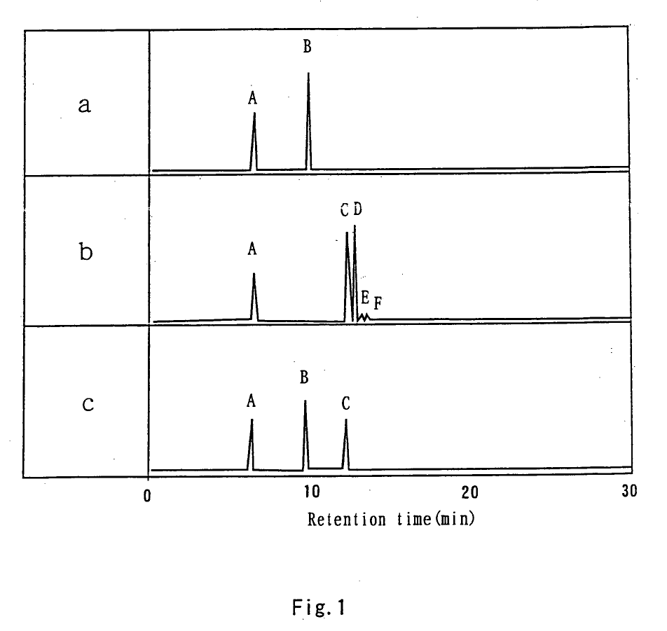

The isomer of conjugated linoleic acid produced by Lactobacillus oris in Example 1 was analyzed. FIG. 1 is a gas chromatographic chart of the isomer of conjugated linoleic acid produced by Lactobacillus oris, showing that all the conjugated linoleic acid produced by Lactobacillus oris is the cis-9, trans-11 isomer of conjugated linoleic acid. The result shows that, among a multitude of lactic acid bacteria, Lactobacillus oris selectively and efficiently produces the cis-9, trans-11 isomer of conjugated linoleic acid in a high content.

### Example 4

### Production of Conjugated Fatty Acids in Fermented Milk by Bacterium Belonging to the Genus Lactobacillus

In 1 mL of a 100 mM phosphate buffer (pH 6.5) were dissolved 50 mg of linoleic acid and 10 mg of BSA to yield a solution of a complex between linoleic acid and BSA. A total of 200 μL of the solution of a complex between linoleic acid and BSA was added to 15 mL of MRS medium (LACTOBACILLI MRS BROTH, a product of DIFCO), and Lactobacillus oris was inoculated thereinto and cultivated at 28° C. for 20 hours with shaking at 120 rpm to yield a cultured mixture of pH 4.7.

To 5 mL of a 10% skim milk medium (supplemented with 1% of glucose and 0.1% of soybean peptide) dispensed in 15-mL test tubes with a cap were added 100 μL of the solution of a complex between linoleic acid and BSA and 200 μL of the above-prepared cultured mixture of the individual strain, and the cap was closed. The medium was cultivated at 28° C. for 48 hours with shaking at 120 rpm to yield a fermented milk. The resulting fermented milk was subjected to analysis on fatty acids by the same procedure of Example 1.

The result shows that 2.0% of the added linoleic acid was converted into a conjugated linoleic acid, and that all the produced conjugated linoleic acid is the cis-9, trans-11 isomer of conjugated linoleic acid, as in Example 3. These results show that Lactobacillus oris selectively and efficiently produces the cis-9, trans-11 isomer of conjugated linoleic acid also in a fermented milk.

### Example 5

### Production of Conjugated Fatty Acid in the Reaction of Washed Cells of Bacteria Belonging to the Genus Bifidobacterium

In 1 mL of a 100 mM phosphate buffer (pH 6.5) were dissolved 50 mg of linoleic acid and 10 mg of BSA to yield a solution of a complex between linoleic acid and BSA.

The solution of a complex between linoleic acid and BSA was added to 15 mL of GAM broth (a product of Nissui Pharmaceutical Co., Ltd.) in a proportion of 0.07%, and Bifidobacterium breve YIT 10001 (FERM BP-8205) was inoculated to the mixture and cultivated at 35° C. for 48 hours with shaking at 120 rpm. The cultured mixture was separated by centrifugation to collect cells, and the cells were washed twice with physiological saline to yield washed cells.

The washed cells were mixed with 100 μL of the solution of a complex between linoleic acid and BSA and 0.9 mL of a 100 mM phosphate buffer (pH 6.5) in a test tube. After replacing the gas phase with nitrogen gas, the test tube was sealed to maintain the inside anaerobic, and a reaction was carried out at 37° C., 120 rpm for 72 hours.

The resulting reaction mixture was mixed with 1 mg of an internal standard (HEPTA-DECANOIC ACID), extracted according to a Bligh-Dyer method, converted into a methyl ester (standing still in a 4% solution of hydrochloric acid in methanol at room temperature for 30 minutes) and analyzed on fatty acids by gas chromatography.

The result shows that Bifidobacterium breve YIT 10001 (FERM BP-8205) converts 0.9% of added linoleic acid in the substrate into the cis-9, trans-11 isomer of conjugated linoleic acid.

### Example 6

### Production of Conjugated Fatty Acid by Dead Cells of Lactobacillus Oris

In 1 mL of a 100 mM phosphate buffer (pH 6.5) were dissolved 50 mg of linoleic acid and 10 mg of BSA to yield a solution of a complex between linoleic acid and BSA. A total of 200 μL of the solution of a complex between linoleic acid and BSA was added to 15 mL of MRS medium (LACTOBACILLI MRS BROTH, a product of DIFCO), and Lactobacillus oris was inoculated thereinto and cultivated at 28° C. for 20 hours with shaking at 120 rpm to yield a cultured mixture of pH 4.7.

To 15 mL of ILS medium supplemented with 0.3% of glucose was added 0.5 mL of the above-prepared cultured mixture, and cultivated at 37° C. for 18 hours (to pH 5.6) with shaking at 120 rpm. Cells were collected by centrifugal separation and washed twice with a 0.2 M glycine buffer (pH 10.6). The washed cells were dissolved in 2 mL of a solution of 6.7% sucrose—50 mM tris (hydroxymethyl) aminomethane—1 mM EDTA. The solution was mixed with 0.8 mL of a lysozyme solution (10 mg/mL in 25 mM tris (hydroxymethyl) aminomethane, pH 8.0, a product of SEIKAGAKU CORPORATION) and 0.15 mL of an aqueous solution of N-Acetylmuramidase (1 mg/mL, a product of SEIKAGAKU CORPORATION) and was reacted at 37° C., 120 rpm for 30 minutes. The reaction mixture was transferred to Centriprep 10 (a product of Amicon Inc.) and concentrated to yield about 0.6 mL of a suspension of dead cells of Lactobacillus oris.

The suspension of dead cells was mixed with 100 μL of the solution of a complex between linoleic acid and BSA and 0.9 mL of a 100 mM phosphate buffer (pH 6.5), and the mixture was reacted in an oxygen-impermeable plastic bag whose atmosphere was maintained anaerobic by using an agent for oxygen absorbing and carbon dioxide gas generating (Anaero Pack, a product of Mitsubishi Gas Chemical Company, Inc.) at 37° C., 120 rpm for 24 hours.

The resulting reaction mixture was subjected to fatty acid analysis by the procedure of Example 1 to find that 1.6% of the added linoleic acid was converted into a conjugated linoleic acid. All the produced conjugated linoleic acid is the cis-9, trans-11 isomer of conjugated linoleic acid, as in the washed cells and the fermented milk. These results show that Lactobacillus oris selectively and efficiently produces the cis-9, trans-11 isomer of conjugated linoleic acid even as dead cells whose cell walls have been broken.

### Example 7

### Production of Conjugated Fatty Acid by Lactobacillus Pontis

In 1 mL of a 100 mM phosphate buffer (pH 6.5) were dissolved 50 mg of linoleic acid and 10 mg of BSA to yield a solution of a complex between linoleic acid and BSA. A total of 200 μL of the solution of a complex between linoleic acid and BSA was added to 15 mL of MRS medium (LACTOBACILLI MRS BROTH, a product of DIFCO), and Lactobacillus pontis (ATCC 51518) was inoculated thereinto and cultivated at 28° C. for 48 hours with shaking at 120 rpm to yield a cultured mixture having a pH of 4.9.

To 5 mL of a 10% skim milk medium dispensed and sterilized in a 15-mL test tube with a cap were added 100 μL of the solution of a linoleic acid-BSA complex and 400 μL of the above-prepared cultured mixture of the strain, and the cap was closed. The medium was cultivated at 28° C. for 48 hours with shaking at 120 rpm to yield a fermented milk having a pH of 5.5. The resulting fermented milk was subjected to analysis on fatty acids by the same procedure of Example 1.

The result shows that 0.5% of the added linoleic acid was converted into a conjugated linoleic acid, and that all the produced conjugated linoleic acid was the cis-9, trans-11 isomer of conjugated linoleic acid. These results show that Lactobacillus pontis selectively and efficiently produces the cis-9, trans-11 isomer of conjugated linoleic acid in a high content.

### Example 8

### Preparation of a Fermented Food Containing a Conjugated Linoleic Acid with the Use of Bifidobacterium Breve YIT 10001 (FERM BP-8205)

A medium containing 10% of skim milk powder, 1% of glucose and 0.1% of soybean peptide was supplemented with 0.1% or 1.0% of linoleic acid and homogenized at 150 kg/cm². The homogenized medium was sterilized in an autoclave at 115° C. for 10 minutes to yield a medium for the preparation of a fermented food.

Bifidobacterium breve YIT 10001 (FERM BP-8205) was inoculated into 15 mL of GAM broth (a product of Nissui Pharmaceutical Co., Ltd.) and cultivated at 35° C. for 48 hours with shaking at 120 rpm to yield a cultured mixture of the bacterium belonging to the genus Bifidobacterium. A total of 6 mL of the cultured mixture was inoculated into 150 mL of the above-prepared medium for the preparation of a fermented food, the gas phase was replaced with nitrogen gas, and the medium was anaerobically cultivated while standing at 35° C., 120 rpm for 72 hours to yield a fermented food.

The resulting reaction mixture was analyzed on fatty acids by the procedure of Example 2 to find that part of the added linoleic acid was converted into a conjugated linoleic acid, and that all the produced conjugated linoleic acid was the cis-9, trans-11 isomer of conjugated linoleic acid.

The prepared fermented food was subjected to an organoleptic test to find to be equivalent to a food containing no linoleic acid.

We claim:

1. A process for producing a conjugated fatty acid, comprising the step of conjugating an unsaturated fatty acid having at least two double bonds, with the use of viable cells, dead cells or a cell extract of at least one bacterium having conjugation capability selected from the group consisting of Lactobacillus oris, Lactobacillus pontis, Lactobacillus panis, Bifidobacterium breve, Bifidobacterium infantis, Bifidobacterium bifidum and Bifidobacterium pseudocatenulatum, or an enzyme derived from the said bacterium.

2. The process according to claim 1, wherein said bacterium having conjugation capability is selected from the group consisting of Lactobacillus oris ATCC 49062, Lactobacillus pontis ATCC 51518, Lactobacillus pontis ATCC 51519, Lactobacillus panis JCM 11053, Bifidobacterium breve YIT 10001 (FERM BP-8205), Bifidobacterium breve ATCC 15698, Bifidobacterium breve ATCC 15701, Bifidobacterium bifidum YIT 4007 (FERM BP-791), Bifidobacterium infantis ATCC 15702 and Bifidobacterium pseudocatenulatum ATCC 27919.

3. The process according to claim 1, wherein said unsaturated fatty acid having at least two double bonds is an linoleic acid.

4. The process according to claim 1, comprising the steps of inoculating the bacterium having conjugation capability into a culture medium which contains the unsaturated fatty acid having at least two double bonds, and cultivating the bacterium in said culture medium.

5. The process according to claim 4, wherein a milk medium is used as the culture medium to yield a fermented milk containing a conjugated fatty acid.

6. The process according to claim 4, wherein a milk medium being free of lipid-binding proteins and surfactants is used as the culture medium to yield a fermented milk containing a conjugated fatty acid.

7. The process according to claim 1, wherein a bacterium being capable of converting linoleic acid into a conjugated linoleic acid is used as the bacterium having conjugation capability, said bacterium being capable of producing a conjugated linoleic acid in an amount exceeding 10, based on 100 of the substrate linoleic acid added to a culture medium, by the following steps:
(a) carrying out a preculture in a growth medium for at least 12 hours, the growth medium comprising a complex between linoleic acid and at least one material selected from BSA, lipid-binding proteins and surfactants; and
(b) inoculating a bacterium having conjugation capability obtained in the step (a) to a milk medium containing linoleic acid and cultivating the bacterium with shaking for a predetermined time.

8. A process for producing a composition of a conjugated linoleic acid enriched for the cis-9, trans-11 isomer, comprising the step of conjugating linoleic acid with the use of viable cells, dead cells or a cell extract of a bacterium having conjugation capability or an enzyme derived from the bacterium to yield a composition containing a conjugated linoleic acid,
wherein said bacterium having conjugation capability is selected from the group consisting of Lactobacillus oris ATCC 49062, Lactobacillus pontis ATCC 51518, Lactobacillus pontis ATCC 51519, Lactobacillus panis JCM 11053, Bifidobacterium breve YIT 10001 (FERM BP-8205), Bifidobacterium breve ATCC 15698, Bifidobacterium breve ATCC 15701, Bifidobacterium bifidum YIT 4007 (FERM BP-791), Bifidobacterium infantis ATCC 15702 and Bifidobacterium pseudocatenulatum ATCC 27919 to thereby yield a conjugated linoleic acid composition enriched for the cis-9, trans-11 isomer, in which most of the conjugated linoleic acid is the cis-9, trans-11 isomer of conjugated linoleic acid.

9. Use of a bacterium in conjugation, the bacterium being capable of producing a conjugated linoleic acid in an amount exceeding 10 based on 100 of the substrate linoleic acid added to a culture medium, through the following steps:
(a) carrying out a preculture in a growth medium for at least 12 hours, the growth medium comprising a complex between linoleic acid and at least one material selected from BSA, lipid-binding proteins and surfactants; and
(b) inoculating a bacterium having conjugation capability obtained in the step (a) to a milk medium containing linoleic acid and cultivating the bacterium with shaking for a predetermined time.

10. The use of a bacterium in conjugation according to claim 9, wherein the bacterium is Lactobacillus oris ATCC 49062 or Bifidobacterium breve YIT 10001 (FERM BP-8205).

11. A food/drink containing a conjugated fatty acid produced by the process according to claim 1.
